(19) United States
(12) Patent Application Publication
(10) Pub. No.: US 2015/0297852 A1
(43) Pub. Date: Oct. 22, 2015

(54) OROPHARYNGEAL CANNULA COMPRISING A DIOXYGEN INLET AND A CARBON DIOXIDE OUTLET

(71) Applicant: DELTAMEDICS, Gouvieux (FR)

(72) Inventor: Jean-Christophe OZENNE, Gouvieux (FR)

(21) Appl. No.: 14/408,724

(22) PCT Filed: Jun. 7, 2013
(86) PCT No.: PCT/EP2013/061826

§ 371 Date: Dec. 17, 2014

(30) Foreign Application Priority Data
Jun. 21, 2012 (FR) 1255869

(51) Int. Cl. A61M 16/04; A61B 5/083; A61B 5/097

(52) U.S. Cl. CPC A61M 16/0493; A61M 16/0497; A61B 5/097; A61B 5/0836; A61M 2202/02; A61M 2230/432

(57) ABSTRACT

Oropharyngeal cannula (1) including a body (2) formed by a distal part (2d) which is curved in order to be inserted into a patient's mouth and to immobilize his tongue and by a proximal part (2p) including a collar (3) which can rest on the patient's lips, a main duct (5) forming a passage for fluid between the proximal part and the distal part of the body, and two auxiliary ducts (6, 7) which are formed in the body and extend from the proximal part (2p) as far as into the main duct. Each auxiliary duct opens into the proximal part of the body over an input/output orifice (64, 74) which is oriented in a direction which is radial relative to the axis of the main duct, such as to be oriented laterally on either side of the patient's mouth when the cannula is installed on the patient.

The invention relates to a device for clearing the airways of a patient, and more particularly an oropharyngeal cannula, allowing injection of dioxygen and also capnographic measurement.

A cannula is a straight or bent tube which permits the passage of a fluid such as air or a liquid through an orifice. An oropharyngeal cannula, such as a Guedel cannula or a Mayo cannula, is used in medicine to keep open the airways of an unconscious patient, for example a patient who is under anaesthetic or a patient who is in a coma.

A Guedel cannula is in the form of a semi-rigid plastic tube comprising three parts.

A first part is curved such as to follow the trajectory of the oral cavity as far as the pharynx. Its form makes it possible in particular to bring forward the lingual mass, spacing the latter from the rear wall of the pharynx, and thus to clear a channel between the patient's tongue and palate, as far as his pharynx. By this means, a passage of air is maintained towards the lungs, and the tongue is held in position so that it does not sink rearwards onto the epiglottis and obstruct the respiratory tracts, in particular when the unconscious patient is lying on his back.

A second, straight part is reinforced in the interior of a rigid ring which is designed to be positioned in the dental area. The rigidity of this area makes it possible to keep the orifice open when the cannula is placed between the teeth, preventing an intubated patient from obstructing an orotracheal tube by biting, for example.

A third part comprises a collar which rests on the patient's lips, once the cannula is in place.

Several sizes of Guedel cannula exist. The sizes vary according to the distance between the corner of the lips and the angle of the jaw in children and adults. In adults, it is also possible to use the distance between the corner of the lips and the earlobe.

During an operation under anaesthetic, a face mask is generally used in order to inject dioxygen into the patient via the duct of the Guedel cannula and the patient's nostrils. However, the use of a mask impedes access to the patient's face, if this is necessary.

In addition, the use of a mask gives rise to complex positioning in order to be able to carry out capnography of the air exhaled by the patient.

Capnography is a measurement of the concentration or partial pressure of carbon dioxide in the air exhaled by a patient. Measurements of this type are very commonly used on patients under anaesthetic. The presence of carbon dioxide in the air exhaled over several exhalations by a patient who has just been intubated makes it possible in particular to confirm that the endotracheal tube is properly placed in the trachea.

Capnography also makes it possible to obtain an indirect measurement of the partial pressure of carbon dioxide in the arterial blood. This information makes it possible to evaluate the state of vascularization of the patient. Capnography reflects directly the capacity of the patient's lungs to eliminate carbon dioxide, and it reflects indirectly the production of carbon dioxide by the tissues and its transport as far as the lungs.

It makes it possible to detect very early signs of respiratory deficiencies such as hypoventilation, or disconnection of a circuit or tube in the oesophagus. During an operation under anaesthetic, capnography makes it possible to provide information such as the frequency and regularity of ventilation, which is more useful than that provided by an oximeter.

It provides a method for rapid detection of critical conditions, such as a badly positioned tracheal tube, a ventilation defect, or a circulatory defect, and for prevention of irreversible complications.

In order to carry out both an injection of dioxygen and collection of the gases exhaled by the patient, so as to carry out a measurement of the level of carbon dioxide, whilst dispensing with the use of a cumbersome face mask, a suitable nasopharyngeal device is known from document US 2007/0095347. However, a device of this type can be used only nasally.

There is also known from documents US 2007/267024, US 2008/308108 and EP 1 188 457 a device which is designed to form a passage for gas to be inhaled, in which there is accommodated a pair of ducts which are designed to be slid into the inner passage(s) in order to inject dioxygen, in particular over a proximal portion, and to collect exhaled gas, in particular over a distal portion, in order to measure the level of CO₂.

However, these devices have the disadvantage of being in several parts, i.e. a part consisting of the cannula, and a part consisting of the injection and extraction tubes to be inserted. The additional assembly time can be critical in particular in an emergency situation.

An oropharyngeal device is also known from document US 2008/0000481, with a form which is designed to be inserted in a patient's mouth. The device comprises a body comprising a distal portion, a proximal portion, and at least two channels which extend from the proximal portion to the distal portion, in order to form two air passages through which firstly dioxygen can be injected, and secondly from which carbon dioxide can be extracted. The channels are formed in the body of the device, which comprises a collar on the proximal portion in order to prevent the device from being introduced too deeply into the mouth.

These oropharyngeal devices do not have means for simple and rapid securing on the patient, which allow them to be maintained on the patient once the device has been installed in the patient's mouth, whilst permitting rapid extraction of the device if the patient wakes up, or if intubation is necessary.

In fact, an oropharyngeal cannula must be installed and secured on the patient such that the cannula can be withdrawn if necessary, in particular if the patient wakes up. The securing of the cannula makes it possible to hold the device in position, but this securing must be easily removable. In fact, Guedel cannulas can cause vomiting in a patient who is conscious. A patient who recovers consciousness will spontaneously cough out the cannula, when he regains the coughing reflex. The cannula can also be replaced at a given moment by an intubation device.

The object of the invention is to eliminate the aforementioned disadvantages, by providing an oropharyngeal cannula which is designed to be secured on the patient, in particular by means of the tubes for injection/extraction of the cannula in place on the patient.

According to one aspect of the invention, an oropharyngeal cannula is proposed comprising a body formed by a distal part which is curved in order to be inserted into a patient's mouth and to immobilize his tongue and by a proximal part comprising a straight portion which is coupled to a collar which can rest on the patient's lips, a main duct forming a passage for fluid between the proximal part and the distal part of the body, and two auxiliary ducts which are formed in the body and extend from the proximal part as far as into the main duct.

According to a general characteristic of the invention, each auxiliary duct opens into the collar over an input/output orifice which is oriented in a direction which is radial relative to the axis of the main duct, such as to be oriented laterally on both sides of the patient's mouth when the cannula is installed on the patient.

By orienting the input/output orifices in this manner, the injection/extraction tubes, which are coupled to the input/output orifices, extend in the direction of the patient's ears, on both sides of the patient's mouth. By this means, each injection/extraction tube can be passed behind an ear, in order to secure the oropharyngeal cannula on the patient in a manner which is easily removable.

Preferably, the direction between each input/output orifice and the axis of the main duct forms an angle of between 0° and 30°, and more particularly between 10° and 20°, with an axis which passes via the corners of the mouth when the cannula is installed on the patient.

An angle of between 10° and 20°, and in particular an angle of 15°, makes it possible to direct the injection/extraction tubes towards the upper edge of the ears, and thus to facilitate the passage of the tubes behind the ears.

The collar can comprise a first and a second input/output bend, which are each integral with an end of an auxiliary duct, each bend comprising one of the said input/output orifices of the collar at the opposite end.

In this embodiment, the collar can have any thickness. The input/output bends project from the surface of the collar.

According to an alternative, each auxiliary duct can comprise a portion which is formed radially in the collar, the input/output orifice being disposed radially on the collar.

In this embodiment, the thickness of the collar must be large enough to permit the passage of the auxiliary ducts.

Advantageously, the oropharyngeal cannula can comprise two flexible injection/extraction tubes which can be passed behind the patient's ears when the cannula is installed on the patient, a first end of each tube being connected to an input/output orifice.

The tubes can be connected to the input/output orifices in a removable manner, or securely and non-removably. The presence of the integral tubes on the oropharyngeal cannula makes it possible to avoid any assembly operation, apart from the connection of the free ends of the tubes to extraction means, such as a capnograph, or to injection means during the installation of the cannula on the patient.

The oropharyngeal cannula can advantageously comprise a clamping ring which can hold the two tubes together and slide along the two tubes such as to hold the cannula in place when it is installed on the patient and each tube passes behind an ear.

Once the tubes have been passed behind each ear, the clamping ring makes it possible to clamp the tube under the chin by raising the clamping ring towards the chin. This makes it possible to ensure secure fastening of the cannula, whilst making it possible to remove the cannula rapidly if necessary.

Advantageously, a second end of the injection/extraction tubes can comprise means for connection to injection or extraction devices.

The connection means can be specific connections which are dedicated to devices for measurement or injection of gas. By having a specific connection for each tube, the risks of connection error are zero, even in an emergency situation, since each connection is dedicated to a specific device.

Preferably, the first auxiliary duct is designed for the injection of dioxygen into the patient's respiratory tracts, and the second auxiliary duct is designed for the extraction of gases exhaled by the patient, in order to carry out a capnographic measurement.

The second auxiliary duct preferably opens into the distal portion and the first auxiliary duct opens into the proximal portion, opposite the collar.

By separating the locations where the auxiliary ducts open into the main duct, the disturbances which are associated in particular with the turbulence created by the injection of dioxygen are reduced for the extraction of the gases exhaled by the patient, and the carbon dioxide measurement offers a better ratio of signal to noise. According to empirical measurements and fluid mechanics, it is preferable to carry out extraction of the gases exhaled in an area which is more distal than that for the injection of oxygen, in order for there to be less disruption by the flow of oxygen.

According to a variant, it is also possible to have two tubes for collection of carbon dioxide which are coupled to the two input/output orifices, the two tubes being coupled to a single capnography device. By this means, the device can be used only for sampling of the carbon dioxide. In this configuration, the two auxiliary ducts can open into the main duct at locations which are similar or separate. Similarly, the oropharyngeal cannula can be used only for the injection of dioxygen via the two auxiliary ducts.

Preferably, the first auxiliary duct is formed in an upper wall of the distal part of the body and the second auxiliary duct is formed in a lower wall of the distal part of the body.

Providing the second auxiliary duct in the lower wall of the distal part reduces the risks of blockage by secretions such as mucus and saliva. In fact, when the patient is lying on his back, the lower surface of the distal wall of the oropharyngeal cannula installed in the patient's mouth is raised to the level of the free end at the back of the throat where the secretions can accumulate.

Each auxiliary duct can advantageously comprise a cross section with an oblong form, at least over the distal part of the body.

By this means, the volume of fluid, and in particular gaseous fluid, which can be conveyed in the duct is larger than in the case of a duct with a cylindrical cross section. In addition, with a body comprising a main duct with an oblong cross section, i.e. which is similar to the form of a mouth, the auxiliary ducts can be provided in the upper wall and the lower wall, without the need to increase substantially the thickness of the walls of the body of the cannula, whilst maintaining a substantial volume of gas transferred.

Advantageously, the cannula can comprise at least one additional duct which is formed in the body and extends from the collar as far as at least the distal part of the body. The additional duct can be designed for aspiration of secretion in the patient's pharynx, so as to prevent the respiratory tracts from being obstructed by these secretions. The secretions can also be aspirated via a tube inserted in the main duct.

The oropharyngeal cannula can also advantageously comprise an additional duct formed in the collar and extending from the first auxiliary duct as far as two additional output orifices provided in an upper part of the collar, the two additional output orifices being disposed such as to be opposite each of the patient's nostrils when the cannula is installed on the patient.

Preferably, at least one of the auxiliary ducts comprises an orifice which opens into the main duct and has a frusto-conical form with a larger cross section than the mean cross section of the auxiliary duct.

The frusto-conical form of the orifice which opens over the main duct at the level of the tubular portion of the body makes it possible to assist good distribution of the dioxygen injected in one case, and to optimize the aspiration of the secretions in another case.

Each portion of auxiliary duct which extends radially in the collar forms an angle of between 0° and 20° and more particularly an angle of approximately 10° relative to the plane defined by the surface of the collar, such as to orient the input/output orifices towards the patient's face.

This angle makes it possible to orient the injection/extraction tubes such as to keep them as close as possible to the patient's face, and thus to reduce the risk of an element or a tool getting caught in the injection/extraction tubes.

Other advantages and characteristics of the invention will become apparent from examination of the detailed description of an embodiment, which is in no way limiting, and of the attached drawings, in which:

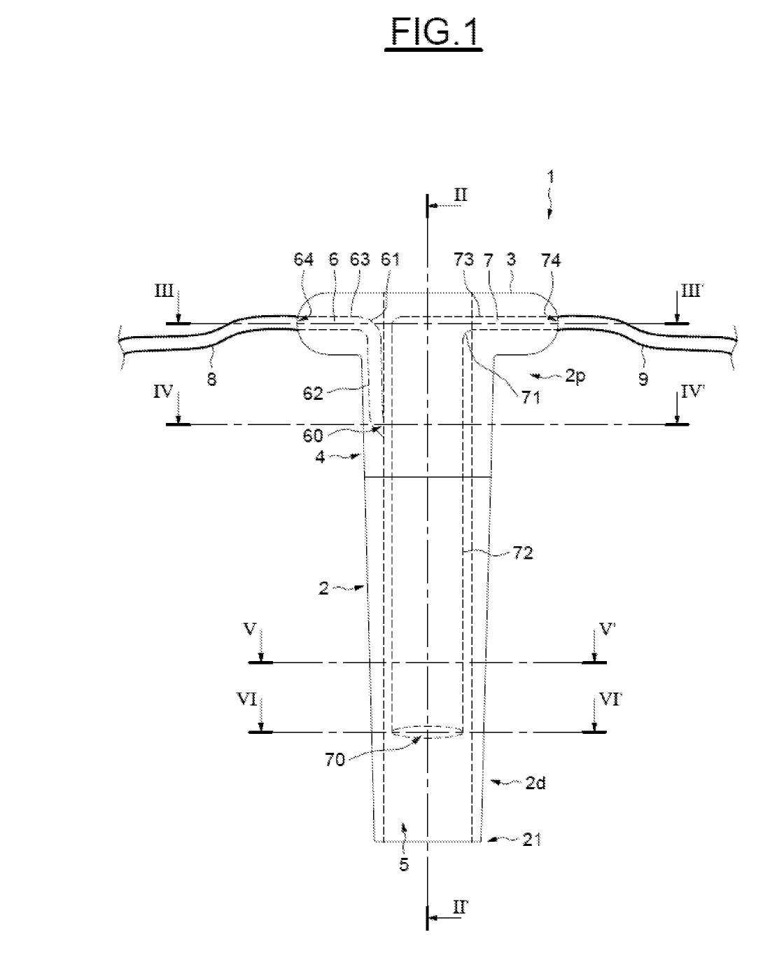

FIG. 1 represents schematically a view from below of an oropharyngeal cannula according to an embodiment of an invention;

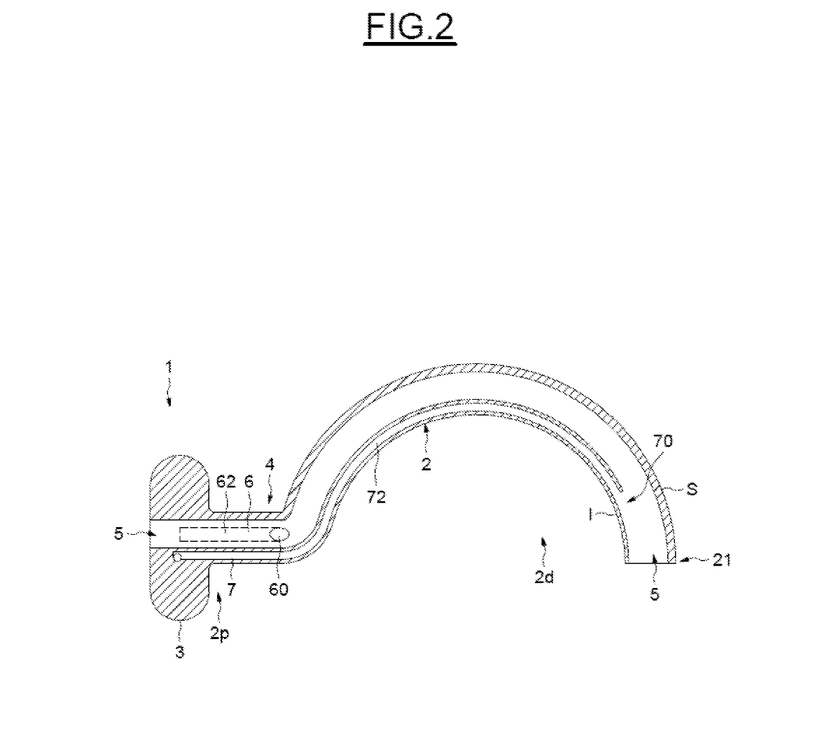

FIG. 2 illustrates a view in cross section in a transverse plane II-II′ of the oropharyngeal cannula in FIG. 1;

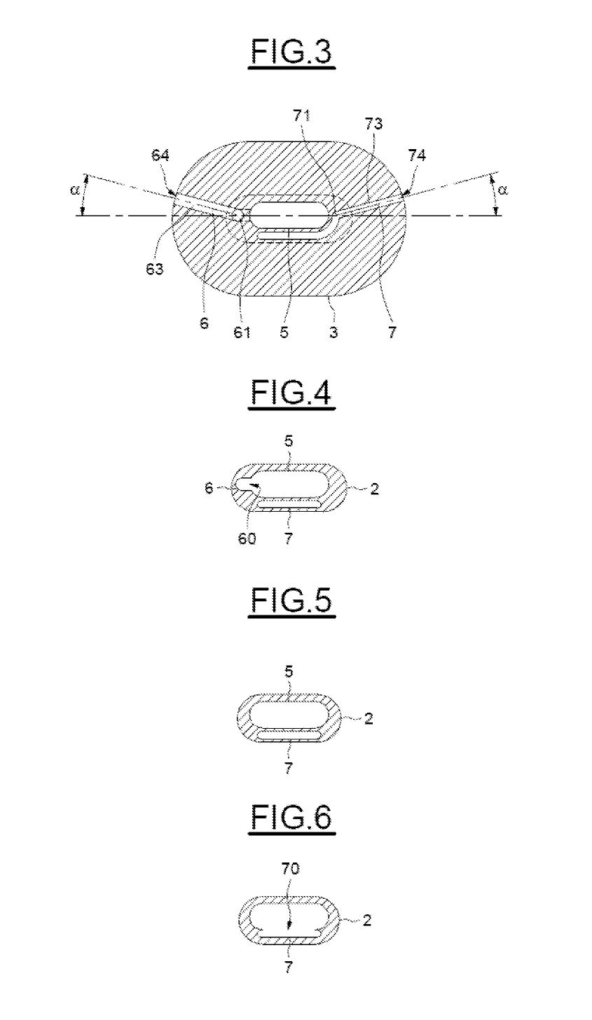

FIG. 3 illustrates a view in cross section in a transverse plane III-III′ of the oropharyngeal cannula in FIG. 1;

FIG. 4 illustrates a view in cross section in a transverse plane IV-IV′ of the oropharyngeal cannula in FIG. 1;

FIG. 5 illustrates a view in cross section in a transverse plane V-V′ of the oropharyngeal cannula in FIG. 1;

FIG. 6 illustrates a view in cross section in a transverse plane VI-VI′ of the oropharyngeal cannula in FIG. 1;

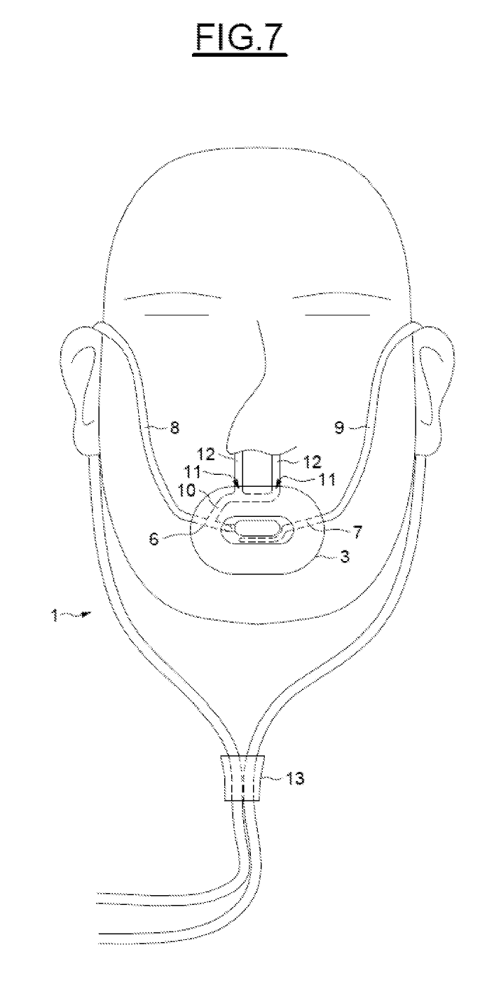

FIG. 7 shows a diagram of an oropharyngeal cannula installed on a patient according to another embodiment.

FIG. 1 represents schematically a view from below of an oropharyngeal cannula 1 according to an embodiment of the invention.

The oropharyngeal cannula comprises a body 2 made of rigid plastic formed by a proximal part 2p and a distal part 2d. As illustrated in FIG. 4, which shows a transverse cross section according to the plane II-II′ of the cannula in FIG. 1, the distal part 2d has a curved form. This part is curved so as to permit the insertion of the oropharyngeal cannula, and in particular its distal part, into a patient's mouth and to immobilize the lingual mass at the front, thus keeping the pharynx open.

The proximal part comprises a collar 3 and a straight portion 4 which is disposed between the collar and the distal part 2d. The collar 3 is designed to rest on the patient's lips once the cannula has been inserted in the patient's mouth, whereas the straight portion 4 is at the level of the patient's teeth. The body comprises a main duct 5 which extends from the collar 3 of the proximal portion 2p as far as the free end 21 of the distal part 2d, whilst passing via the straight portion 4. The main duct 5 thus forms a fluid passage between the proximal part 2p and the distal part 2d of the body 2 of the oropharyngeal cannula 1. Once installed on this patient, the main duct 5 forms a fluid passage between the patient's pharynx and lips.

The straight portion 4 can be made of a material which is more rigid than the rest of the body 2 of the cannula 1, so as to reinforce the area against any risk of biting, which could give rise to obstruction of the main duct 5. In order to reinforce the straight portion 4, a sleeve, not represented, can also be inserted in the main duct 5 through the collar 3 so as to extend between the collar 3 and the straight portion 4, the sleeve being hollow in its central axis in order to maintain a fluid passage in the main duct 5.

As illustrated in FIGS. 1 and 2, the oropharyngeal cannula 1 comprises a first auxiliary duct 6 formed in the thickness of the body 2, which separates the main duct 5 from the exterior of the body 2. The first auxiliary duct 6 extends from the collar 3 at the end of the straight portion 4 which is connected to the distal part 2d. It opens into the main duct 5 via a first opening 60, as represented in FIG. 1, and also in FIG. 4, which represents a view in the cross-sectional plane IV-IV′ of the oropharyngeal cannula 1 in FIG. 1.

As represented in FIG. 1 and in FIG. 3, which represents a view in cross section in the plane III-III′ of the oropharyngeal cannula 1 in FIG. 1, the first auxiliary duct 6 comprises a bend 61 which connects a first portion 62 of the first auxiliary duct 6, which extends parallel to the axis II-II′ as far as the first opening 60, and a second portion 63 of the first auxiliary duct 6, which extends into the collar 3. The second portion 63 extends into the collar 3 according to a direction which is radial relative to the axis II-II′, as far as an input orifice 64.

The oropharyngeal cannula 1 comprises a second, auxiliary duct 7 which is also formed in the thickness of the body 2, and has a form with a cross section which is oblong. The second auxiliary duct 7 passes through the body 2 from the collar 3, and opens into the main duct 5 via a second opening 70, which is disposed upstream from the free end 21 of the distal part 2d, as represented in FIGS. 1 and 2, and also in FIG. 6, which represents a view in the cross-sectional plane VI-VI′ of the oropharyngeal cannula in FIG. 1. Positioning the second opening 70 thus upstream from the free end 21 of the distal portion reduces the risks of obstruction of the second opening 70 by secretions in the throat.

These risks are limited further still by the choice of providing the second auxiliary duct 70 in the lower wall I of the distal part 2d. In fact, when the patient is lying on his back, the lower wall I of the distal wall 2d of the oropharyngeal cannula 1 installed in the patient's mouth is raised to the level of the free end 21 at the back of the throat where the secretions can accumulate.

In order to reduce all the more any accumulation of secretions in the throat, the oropharyngeal cannula 1 can comprise an additional duct, not represented, which is formed in the body 2 and extends from the collar 3 to the free end 21 of the distal part 2d. The additional duct is preferentially produced in the upper wall S of the distal part 2d, such as to be in contact with the back of the throat where the secretions accumulate. The additional duct is coupled to aspiration means, so as to eliminate the secretions.

As represented in FIGS. 1 to 3, the second auxiliary duct 7 comprises a bend 71 which connects a first portion 72 of the second auxiliary duct 7 which extends according to the axis II-II′, as far as the second opening 70, and a second portion 73 of the second auxiliary duct 7 which extends in the collar 3. The second portion 73 of the second auxiliary duct 7 extends in the collar 3 according to a direction which is radial relative to the axis II-II′ as far as an output orifice 74.

The first auxiliary duct 6 is designed to supply a flow of dioxygen. The second auxiliary flow 7 is designed to collect a portion of the gases exhaled by the patient, in order to measure the level of carbon dioxide in the gases exhaled by means of a capnograph.

For this purpose, the input orifice 64 and the output orifice 74 are connected respectively to an injection tube 8 and an extraction tube 9 as illustrated in FIG. 1. The connection is produced by welding or by over-moulding the collar 3 onto the injection and extraction tubes 8 and 9, or by means of a connection component.

The free end of the injection tube 8 comprises a specific connection component, for example a standard conical connection for dioxygen, which is designed to be coupled to a device which provides a flow of dioxygen, optionally via a tube cross section reducer. On the other hand, the free end of the injection tube 9 comprises a specific connection component, for example a connection of the “Luer lock” type, which is designed to be coupled to a capnograph.

As illustrated in FIG. 3, the second portion 63 of the first auxiliary duct 6 and the second portion 73 of the second auxiliary duct 7 each form an angle α with an axis which passes via the first bend 61 and the second bend 62. The angle α has a value of approximately 15°. This angle allows the injection tube 8 and the extraction tube 9 each to be oriented in the direction of the upper edge of an ear of the patient, such as to facilitate the passage of the injection and extraction tubes 8 and 9 behind the ears, as illustrated in FIG. 7.

In addition, the second portion 63 of the first auxiliary duct 6 and the second portion 73 of the second auxiliary duct 7 can each form an angle of approximately 10° relative to the plane defined by the surface of the collar 3, such as to orient the injection and extraction tubes 8 and 9 towards the patient's face, and thus optimize the maintenance in position of the oropharyngeal cannula 1. FIG. 7 shows a diagram of an oropharyngeal cannula installed on the patient according to another embodiment. The passage of the injection and extraction tubes 8 and 9 behind the patient's ears makes it possible to hold the oropharyngeal cannula 1 in place on the patient's lips. In this embodiment, the oropharyngeal cannula 1 additionally comprises a clamping ring 13 which is coupled to the injection and extraction tubes 8 and 9. The clamping ring 13 is fitted such as to slide along the injection and extraction tubes 8 and 9 to make it possible to raise the ring 13 until the injection and extraction tubes are clamped under the patient's chain, so that the oropharyngeal cannula 1 is held securely in position on the patient.

In this embodiment, the cannula 1 also comprises additional outputs for injection of dioxygen nasally. The cannula 1 comprises an additional duct 10 formed in the collar 3. The additional duct 10 is connected to the first auxiliary duct 6, and extends in the collar as far as two additional output orifices 11. The additional output orifices 11 are provided in a radial upper part of the collar 3, such as to be opposite the patient's nostrils when the cannula 1 is installed. The two additional output orifices 11 are coupled to two short additional tubes 12 which are inserted in the patient's nostrils when the cannula 1 is installed, in order to inject dioxygen into the patient's nostrils.

In order to improve the discharge of the moisture which is present in the extraction tube 9, the extraction tube can comprise a portion a few centimetres long, 5 cm for example, of Nafion tube. This portion is preferably situated 2 cm from the collar 3.

The invention proposed provides an oropharyngeal cannula which can be installed and secured rapidly on the patient in a removable manner, whilst being held securely in position on the patient. In addition, the oropharyngeal cannula proposed is produced as a single piece, or is preassembled, such that all that remains is to connect the injection/extraction tubes to the recipient devices. This makes it possible to reduce the installation time and the risks of connection error.

1. Oropharyngeal cannula (1) comprising a body (2) formed by a distal part (2d) which is curved in order to be inserted into a patient's mouth and to immobilize his tongue and by a proximal part (2p) comprising a collar (3) which can rest on the patient's lips, a main duct (5) forming a passage for fluid between the proximal part (2p) and the distal part (2d) of the body (2), and two auxiliary ducts (6, 7) which are formed in the body (2) and extend from the proximal part (2p) as far as into the main duct (5), characterized in that each auxiliary duct (6, 7) opens into the collar (3) over an input/output orifice (64, 74) which is oriented in a direction which is radial relative to the axis (II-II′) of the main duct (5), such as to be oriented laterally on either side of the patient's mouth when the cannula (1) is installed on the patient.

2. Cannula (1) according to claim 1, wherein the direction between each input/output orifice (64, 74) and the axis of the main duct (5) forms an angle of between 0° and 30°, and more particularly between 10° and 20°, with an axis which passes via the corners of the lips when the cannula (1) is installed on the patient.

3. Cannula (1) according to claim 1, wherein each auxiliary duct (6, 7) comprises an input/output bend which is coupled to the collar (3), each auxiliary duct (6, 7) passing through the collar (3) as far as into the input/output bend which opens over the input/output orifice (64, 74).

4. Cannula (1) according to claim 1, wherein each auxiliary duct (6, 7) comprises a portion (63, 73) which is formed radially in the collar (3), the input/output orifice (64, 74) being disposed radially on the collar (3).

5. Cannula (1) according to claim 1, comprising two flexible injection/extraction tubes (8, 9) which can be passed behind the patient's ears when the cannula (1) is installed on the patient, a first end of each tube being connected to an input/output orifice (64, 74).

6. Cannula (1) according to claim 5, comprising a clamping ring which can hold the two tubes (8, 9) together and slide along the two tubes (8, 9) such as to hold the cannula (1) in place when it is installed on the patient and each tube (8, 9) passes behind an ear.

7. Cannula (1) according to claim 5, wherein a second end of the injection/extraction tubes (8, 9) comprises means for connection to injection or extraction devices.

8. Cannula (1) according to claim 1, wherein the first auxiliary duct (6) comprises an input orifice (64) which is connected to a tube (8) for injection of dioxygen into the patient's respiratory tracts, and the second auxiliary duct (7) comprises an output orifice (74) which is connected to a tube (9) for extraction of gases exhaled by the patient, in order to carry out a capnographic measurement.

9. Cannula (1) according to claim 8, wherein the second auxiliary duct (7) opens into the distal portion (2d) and the first auxiliary duct (6) opens into the proximal portion (2p), opposite the collar (3).

10. Cannula (1) according to claim 1, wherein the first auxiliary duct (6) is formed in an upper wall (S) of the distal part (2d) of the body (2) and the second auxiliary duct (7) is formed in a lower wall (I) of the distal part (2d) of the body (2).

11. Cannula (1) according to claim 1, wherein the second auxiliary duct (7) comprises a cross section with an oblong form, at least over the distal part (2d) of the body (2).

12. Cannula (1) according to claim 8, comprising an additional duct (10) formed in the collar (3) and extending from the first auxiliary duct (6) as far as two additional output orifices (11) provided in an upper part of the collar (3), the two additional output orifices (11) being disposed such as to be opposite each of the patient's nostrils when the cannula (1) is installed on the patient.

13. Cannula (1) according to claim 1, wherein at least one of the auxiliary ducts (6, 7) comprises an orifice which opens into the main duct (5) and has a frusto-conical form, the orifice having a larger cross section than the mean cross section of the auxiliary duct (6, 7).

14. Cannula (1) according to claim 4, wherein each portion (63, 73) of auxiliary duct (6, 7) which extends radially in the collar (3) forms an angle of between 0° and 20° and more particularly an angle of approximately 10° relative to the plane defined by the surface of the collar (3), such as to orient the input/output orifices (64 and 74) towards the patient's face.

15. Cannula (1) according to claim 6, wherein a second end of the injection/extraction tubes (8, 9) comprises means for connection to injection or extraction devices.
